(19) United States
(12) Patent Application Publication
(10) Pub. No.: US 2007/0244195 A1
(43) Pub. Date: Oct. 18, 2007

(54) Treatment methods with peroxides and tertiary amines

(76) Inventors: Craig N. Burkhart, Chapel Hill, NC (US); Craig G. Burkhart, Toledo, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 11/709,325

(22) Filed: Feb. 21, 2007

Related U.S. Application Data
(63) Continuation-in-part of application No. 10/847,906, filed on May 18, 2004.
(60) Provisional application No. 60/775,220, filed on Feb. 21, 2006.
(60) Provisional application No. 60/776,095, filed on Feb. 23, 2006.

(51) Int. Cl. A61K 31/192

(52) U.S. Cl. 514/568

(57) ABSTRACT

This invention relates to methods of increasing the efficacy of peroxides such as benzoyl peroxide in the treatment of skin conditions such as acne. In a preferred embodiment, the invention relates to methods of increasing radicals formed by peroxides on/in the skin, more specifically near/in the comedone, for topical use in dermatology. In a specific embodiment, the invention relates to the use of transitional metals such as Cu(l) and ferrous ions to increase the efficacy of peroxides such as benzoyl peroxide. In another embodiment, the invention relates to a method by which a peroxide such as benzoyl peroxide and its activator are added to the skin surface at the same time. In another embodiment, the invention relates to the use of a more soluble form of peroxide such as benzoyl peroxide to increase its efficacy. In another embodiment, the invention relates to the addition of a side chain to a peroxide such as benzoyl peroxide so that it is activated by light. In a further embodiment, the invention relates to the addition of a tertiary amine to a peroxide such as benzoyl peroxide at the time of skin application, to improve the efficacy of the peroxide. In another embodiment, the invention relates to the addition of dapsone or other material to a peroxide such as benzoyl peroxide to improve its efficacy.

### CROSS-REFERENCE TO RELATED APPLICATION

This application is a continuation-in-part of copending U.S. patent application Ser. No. 10/847,906 filed on May 18, 2004, which is incorporated by reference. This application also incorporates by reference U.S. patent application Ser. No. 10/917,240 filed on Aug. 12, 2004, U.S. provisional patent application Ser. No. 60/775,220 filed on Feb. 21, 2006, and U.S. provisional patent application Ser. No. 60/776,095 filed on Feb. 23, 2006.

### BACKGROUND OF THE INVENTION

This invention relates in general to methods of treating skin conditions such as acne, and in particular to methods of increasing the efficacy of peroxides such as benzoyl peroxide in the treatment of skin conditions.

The pathophysiology of acne vulgaris, the most common cutaneous disease, is the consequence of the interplay of follicular hyperkeratinization, bacteria in the follicular canal, and sebum production. The exact mechanism triggering the development of the comedone and the stimuli causing the non-inflamed lesion to become provoked are poorly understood. The microbiology of acne vulgaris and its immunologic ramifications constitute a major thrust of present research in the elucidation of the pathogenesis of inflammatory acne. Within the microbial flora of the pilosebaceous unit, P. acnes is the most meaningful organism in acne causation.

The methods of acne therapy are usually grouped into several categories such as keratolytics, antibacterials, sebosuppressives, and hormones. Benzoyl peroxide (BP) is the most widely used topical agent for acne since its introduction in the 1960's. BP is very effective for the treatment of acne because it is antibacterial, functions as a peeling agent, has comedolytic activity, and reduces free fatty acid levels. Concomitant topical treatment of BP and erythromycin is stated to be superior to BP alone. However, no synergistic activity has been found with this combination. Instead, such combination therapies are hypothesized to gain their efficacy by the coupled action of two effective treatments.

### SUMMARY OF THE INVENTION

This invention relates to methods of increasing the efficacy of peroxides such as benzoyl peroxide in the treatment of skin conditions such as acne. In a preferred embodiment, the invention relates to methods of increasing radicals formed by peroxides on/in the skin, more specifically near/in the comedone, for topical use in dermatology.

In a specific embodiment, the invention relates to the use of transitional metals such as Cu(l) and ferrous ions to increase the efficacy of peroxides such as benzoyl peroxide.

In another embodiment, the invention relates to a method by which a peroxide such as benzoyl peroxide and its activator (or adjunctive agent) are added to the skin surface at the same time (and not days or months before). This ensures that the ingredients are not inactivated or lost strength by being placed together prior to usage.

In another embodiment, the invention relates to the use of a more soluble form of peroxide such as benzoyl peroxide to increase its efficacy.

In another embodiment, the invention relates to the addition of a side chain to a peroxide such as benzoyl peroxide so that it is activated by light.

In a further embodiment, the invention relates to the addition of a tertiary amine to a peroxide such as benzoyl peroxide at the time of skin application, to improve the efficacy of the peroxide. This could include any tertiary amine structure except for an erythromycin structure.

In another embodiment, the invention relates to the addition of dapsone or other material to a peroxide such as benzoyl peroxide to improve its efficacy.

Various advantages of this invention will become apparent to those skilled in the art from the following detailed description of the preferred embodiments.

### DETAILED DESCRIPTION OF THE PREFERRED EMBODIMENTS

This invention relates to methods of increasing the efficacy of peroxides such as benzoyl peroxide in the treatment of skin conditions such as acne. In a preferred embodiment, the invention relates to methods of increasing radicals formed by peroxides on/in the skin, more specifically near/in the comedone (but not limited thereto), for topical use in dermatology. The methods use the radicals formed by peroxides such as benzoyl peroxide, optimizing conditions such that the skin/comedone is the only place they are formed as opposed to in a storage container or wherever the benzoyl peroxide happens to be from the time of application to when the benzoyl peroxide breaks down into its radicals or is metabolized).

The methods of the invention may use the principles of photodynamic therapy directed at acne. Instead of forming radicals in cancer cells, the methods form radicals in/by the comedone (skin surface, sebum within P. acnes). Location and timing of formation of radicals is a very important part of the methods.

The methods use the assumption that radicals derived from BP or other peroxides are the most useful in acne therapy (as opposed to reactive oxygen intermediates used in photodynamic therapy).

In a specific embodiment, the invention relates to the use of transitional metals such as Cu(l) and ferrous ions to increase the efficacy of peroxides such as benzoyl peroxide. The use of transitional metals such as Cu(l) and ferrous ions (as alluded to in the text) to increase the efficacy of benzoyl peroxide. It is anticipated that such an addition to benzoyl peroxide would increase the generation of benzoyloxyl radicals.

The transitional metals include all the elements between Group IIA and IIIa in the periodic table. The list includes zinc, cadmium, mercury, scandium, titanium, vanadium, chromium, manganese, yttrium, zirconium, niobium, molybdenum, technetium, ruthenium, rhodium, palladium, silver, lanthanum, hafnium, tantalum, tungsten, rhenium, osmium, iridium, platinum, gold, mercury, actinium, unnilquadium, unnilpentium, unnilhexium, and uniseptium.

A few characteristics of transitional metals include:
most are harder and more brittle with higher melting points, boiling points, and heats of vaporization than the non-transitional metals.
their ions and compounds are usually colored.
they form many complex ions.
most exhibit multiple oxidation states.
many of them are paramagnetic, as are many of their compounds.
many of the metals and associated compounds are effective catalysts.

In another embodiment, the invention relates to a method by which a peroxide such as benzoyl peroxide and its activator (or adjunctive agent) are added to the skin surface at the same time (and not days or months before). An example of such would be a better package system in which the various ingredients that would be added to benzoyl peroxide would be put into a dispenser with two or three chamber (depending upon the number of items combined) to separate the product's ingredients so they do not interact until the instant you apply them to one's acne. This separation would ensure that the ingredients are not inactivated or lost strength by being placed together prior to usage.

Another example of such a system would be benzoyl peroxide (bp) dissolved in a hydrophobic solvent and the activator in a polar solvent. The BP and activator wouldn't meet until applied onto the skin surface. Lipophilic carriers are well known in the art. For an example of the activator in a hydrophilic solvent, both protic and aprotic solvents are included. Protic solvents such as methanol, ethanol, formamide, N-methylformamide, and water, a hydrogen is attached to the electronegative part of the reagent. The hydrogen has a proton-like character and strongly reacts with anionic nucleophiles. Aprotic solvents do not contain positively polarized hydrogens. These include acetone, acetonnitrile, N,N-dimethylformamide, DMSO, hexamaethylphophoric triamide—the aprotic solvents increase the reactivity of nucleophiles in SN2 reactions (the possible mechanism of radical formation by the BP tertiary amine combination).

Retin A micro is an example of a product released by a polymer. The retin A is stored in a small polymer bead. After application of these beads onto the skin, retin A slowly diffuses out of the polymer and into the skin. The invention would have the activator of benzoyl peroxide radical formation contained in a similar polymer. The activator would be slowly released (by diffusion or breakdown of the polymer) into the skin allowing it to react with BP. Alternatively, the BP could be stored in and released from the polymer. Or, both the activator and BP could be released from their own individual polymers to react when the meet (in the environment of the skin/comedone).

In another embodiment, the invention relates to the use of a more soluble form of peroxide such as benzoyl peroxide to increase its efficacy. The use of a more soluble form of benzoyl peroxide. The present-day products actually use benzoyl peroxide in the form of crystals. We are able to solubilize benzoyl peroxide either by altering its hydric solvents, or by adding a side chain to its structure.

In another embodiment, the invention relates to the addition of a side chain to a peroxide such as benzoyl peroxide so that it is activated by light. We could also add a side chain to benzoyl peroxide so that it is activated by light.

In a further embodiment, the invention relates to the addition of a tertiary amine to a peroxide such as benzoyl peroxide at the time of skin application, to improve the efficacy of the peroxide. This could include any tertiary amine structure except for an erythromycin structure. We believe that benzoyl peroxide efficacy can be improved by adding a tertiary amine at the time of skin application. Therefore, we would be including all substances (and chemicals) which have a tertiary amine within the provisional patent, be they antibiotics or whatever. The invention would include all tertiary amine structures, save for the erythromycin structure that is presently used in a commercial product named benzymycin.

Some nonlimiting examples of tertiary amines include Alfuzosin, Alimemazine, Analgesic drug (Reference 97), Atropine, alpha,alpha-bis [3-(N-benzyl-N-methyl-carbamoyl)-piperidino]-p-xylene dihydrobromide, Bupivacaine, cis-trans-Cavinton, Cloperastine, Cyamemeazine, Cyclopentolate, 2-(4,5-dihydro-1H-imidazol-2-yl)-2-propyl-1,2,3,4-tetrahydropyrrolo ]3,2,1-hi[-indole, 1-decyl-3-(N,N-diethylcarbamoyl) piperidine hydrobromide, Diltiazem, Dimethindene, Diperodone, Disopyramide, Disopyamide, semipreparative, Dixyrazine, Doxazosin, Dropropizine, Hydroxychloroquine and metabolites, Ketoconazole, Laudanosine, Marcaine, Medetomidine, Mepivacaine, Mepivacaine (micro column), Meptazinol, Methadon, Nefopam, Nicotine, Omeprazole, Oxybutynin, Oxyphencyclimide, Pheniramine, 3-PPP, Procyclidine, Promethazine, Proxyphylline, Remoxipride, Tetrahydrozoline, Tetramisole, Tetramisole (micro column), Thioridazine ring-sulphoxide, Tolperisone, Trihexyphenidyl, Trimipramine, Tropicamide, Vamicamide, Verapamil, and Vinca alcaloids. The structures and other characteristics of these tertiary amines can be found on the internet at www.chromtech.se/tertiary.htm. The listed amines are all drugs, but the methods of the invention are not limited to just drugs—any tertiary amine would work.

Along with transition metals, tertiary amines potentiate radical formation by BP. A possible mechanism involves reaction of the amine and BP by a S_{N}2 mechanism. The intermediate thus formed thermally decomposes to benzoyloxy radicals and an amine radical cation. The benzoyloxy radicals may further decompose into phenyl radicals. All of these radicals can react with biological molecules possibly causing some biological effect.

In another embodiment, the invention relates to the addition of dapsone to a peroxide such as benzoyl peroxide to improve its efficacy. Heme is a protoporphyrin. P. acnes actually produces protoporphyrins. 5-aminolevulinic acid (ALA) increases protoporphyrin production by P. acnes. ALA is the same stuff used in photodynamic chemotherapy and photodynamic antimicrobial chemotherapy. Methylene blue, toluidine blue O, phthalocyanine, and haematoporphyrin derivative could also be used. Phenothiazinium dyes could also be used. These materials might work by depleting the antioxidant levels in/around the comedone allowing the BP derived radicals to reach the comedone or spread further throughout the comedone.

Viagra (sildenafil) increases NO production by blood vessels (and maybe the skin). It is an example of a molecule inducing the skin to produce a benzoyl peroxide activator.

Testing and Discussion:

Objective: The purpose was to compare radical activity of BP alone and with various antibiotics to determine whether BP and antibiotics may be chemically synergistic.

Methods: Polymerization of tetra ethylene glycol dimethacrylate was used as a test of BP radical activity. Solutions of BP, antibiotics, and BP and antibiotics were made at 3% w/w in tetraethylene glycol dimethacrylate. All of the antibiotics except erythromycin (ERY) were obtained from prescription pills, which were crushed in a crucible. The portion of the pills that disolved in tetraethylene glycol dimethacrylate were used in the experiment. ERY was obtained in powdered form from Benzamycin® acne treatments. Aliquots of ten drops of these solutions were placed in an eight well plastic plate. The samples were heated in an oven that maintained a temperature range between 90 to 100 degrees Celsius. After various amounts of time the samples were taken out of the oven and tested for gel formation. Polymerization of tetraethylene glycol dimethacrylate was detected visually by swirling a spatula in the solutions. Gelling constituted an indicator of BP radical activity.

Results: The results suggest that radical activity increases upon addition of certain antibiotics, such as erythromycin, to a solution of BP. ERY, minocycline (Vectrin®), and levofloxacin (Levaquin®) in combination with BP caused the tetraethylene glycol dimethacrylate to polymerize the fastest. This is assumed to be due to elevated BP radical formation. Agents that did not augment BP radical activity included doxycycline (Monodox®), and trovofloxacin (Trovan®). Upon storage in a dark room at room temperature, the ERY-BP combination gelled within an hour. The Vectrin®-BP, Diflucan®-BP, Trovan®-BP, Monodox®-BP, and Levaquin®-BP combinations did not gel within six hours. Zithromycin® (a prescription drug containing a macrolide similar to ERY) in combination with BP also gelled within an hour when stored in a dark room at room temperature. Furthermore, Zithromycin®-BP and ERY®-BP solutions gelled within an hour when stored in a refrigerator. Zithromycin® has not been tested yet at higher temperatures.

Discussion: BP induces a variety of biological effects. BP can inhibit metabolic cooperation, alter protein synthesis, induce omithine decarboxylase activity, cause DNA strand breaks, suppress DNA synthesis, and may interfere with mitochondrial respiration. Several of these effects, such as DNA strand breaks, may be caused by BP-derived radicals. Thus, acne treatments that increase the radical activity of BP may be more effective.

Tertiary amines potentiate radical formation by BP. A possible mechanism involves reaction of the amine and BP by a S_{N}2 mechanism. The intermediate thus formed thermally decomposes to benzoyloxy radicals and an amine radical cation. The benzoyloxy radicals may further decompose into phenyl radicals. All of these radicals can react with biological molecules possibly causing some biological effect. Of the antibiotics tested, ERY, doxycycline (Monodox®), minocycline (Vectrin®), levofloxacin (Levaquin®), and trovofloxacin (Trovan®) contain tertiary amines. ERY-BP, Levaquin®-BP, and Vectrin®-BP combinations all behaved as would be expected as they demonstrated faster kinetics for radical formation than BP alone.

Contaminants and solubility may have caused the unexpected results from the Monodox®-BP and Trovan®-BP combinations. The extra chemicals contained in the pills may have dissolved in the tetraethylene glycol dimethacrylate and acted as plastisizers or radical scavengers, thus, hiding any enhanced radical formation by the antibiotic-BP combination. On the other hand, the contaminants may have accelerated the formation of BP-derived radicals. The contaminants may have affected the results for the Levaquin®-BP and Vectrin®-BP combinations as well. Furthermore, some of the antibiotics may not have dissolved in the tetraethylene glycol dimethacrylate, thus, preventing them from being involved in the experiment as only dissolved material was transferred to the plastic plate for testing.

The most impressive result was the speed that the ERY-BP and Zithromycin®-BP solutions gelled at room temperature and below. The speed of reaction between the macrolides and BP insinuates that all the BP in Benzamycin® may be completely depleted by the time a patient picks up his/her prescription to the time it is applied to his/her body. As Benzamycin® is a very effective drug for the treatment of acne, a novel drug may be formed as a product of reactions of BP and ERY with each other and/or other components in Benzamycin® that is very effective against acne. Finding this chemical may result in the discovery of improved acne treatments that do not require BP. As Zithromycin® similarly increased BP radical formation, it is probable that many macrolides mixed with BP are effective drugs for the treatment of acne.

It may be true that the BP is protected from ERY while stored in its container. For example, much of BP is in a less reactive crystalline form while in acne creams, where as it was fully dissolved in these experiments. Upon application to the skin these crystals of BP may dissolve and react with ERY producing radicals. Depending on where these radicals are formed DNA strand breaks, lipid peroxidation, or other effects may occur.

Conclusion: Radical activity of BP in tetraetylene glycol dimethacrylate is increased when tested in consort with several antibiotics, such as the macrolides. We propose that the tertiary amines contained on certain antibiotics are responsible for catalysis of BP radical formation. If increased radical formation correlates with enhanced biological effect, then these data reveal the possibility of biological synergism in mixtures of BP and antibiotics. An understanding of the mechanism of catalysis of BP radical formation by antibiotics may lead to the discovery of improved treatments for acne.

In another embodiment of the invention, liquid nitrogen can be used with certain topical agents that are usually applied in their crystalline state. With freezing, the crystals will fracture causing the agent to be more accessible for reactivity on rewarming. In the case of benzoyl peroxide, the crystals would crack and this would allow more surface area and more benzoyl peroxide to react with other topical agents like tertiary amines.

In a further embodiment of the invention, lasers and all of the various forms of energy, could be used to augment the activity of numerous topical agents. This includes all forms of products and technologies in the treatment of soft tissues via all energy-based modalities including cryogenic energy, hydraulic energy, laser energy, magnetic energy, mechanical energy, microwave energy, radiation energy, radiofrequency energy, thermal energy, vibrational medicine, and ultrasonic energy. We will use the example of lasers, realizing the patent addresses all form of light therapy including light emitting diodes, as well as all forms of energy (because all forms can aid in exciting the reaction with benzoyl peroxide).

Presently, lasers are not used with any topical agents save for:
anesthetic agents to reduce the pain for the patient;
topical indocyanine green photodynamic therapy, visudine-photodynamic therapy (based on benzoporphyrin) and aminolevulinic acid-photodynamic therapy (based on bacterial porphyrins). Photodynamic therapy involves the selective retention of a photosensitizer that upon activation with light mediates tumor cell destruction (photothermolysis) via the production of singlet oxygen. These are not normal, topical medications used for skin conditions but are chromophores, which selectively absorb a monochromatic laser pulse of appropriate wavelength and duration.

One reason why there hasn't been any topical medication used with laser therapy is because of concern that one should not apply anything on the surface that might lead to reflection or reduction of penetration of the laser light.

However, not only may lasers (and other forms of light therapy) be used in consort with topical medications; they can excite or activate certain topical agents for improved therapeutic function and advantage. In such a case, the laser (and other forms of light therapy such as light emitting diodes) would have additive, or possibly synergistic biological effects when used in concert with topical agents. Thus, one may find that one can obtain improved results by putting medications on to the skin surface either prior and/or during and/or after laser therapy for optimum results. This idea applies to all lasers and other forms of light therapy, such as light emitting diodes.

An example of using a medication with laser therapy would be with benzoyl peroxide. Presently, benzoyl peroxide is applied in a crystal state in topical formulations. With the addition of lasers (and other forms of light therapy such as light emitting diodes), one would activate the benzoyl peroxide by heat and/or by breaking the molecule down from its crystalline state. Benzoyl peroxide in solution form is more active with higher antibacterial properties. Heat from the laser (and other forms of light therapy such as light emitting diodes) would also aid in the reaction of benzoyl peroxides with all other chemicals including the tertiary amines. Thus, the lasers would activate the benzoyl peroxide to form benzoyl peroxide radicals that would prove helpful for numerous dermatological states including acne, tinea, wounds, rosacea, precancers, tumors, methicillin-resistant staph aureus, soaps, and cleansers. It may even have benefit for cutaneous cancer. Of note, these radicals are extremely short-lived, and by activating the product on the skin surface, one insures that all the radical formation is on the skin surface for full effectiveness of the benzoyl peroxide and rapid suppression of the dermatological condition. There are other topicals which are tertiary amines (such as minoxidil and diphenhydramine) which react with benzoyl peroxide, and offer benefit when stimulated by laser.

Somewhat similarly, but using a different energy-based modality, liquid nitrogen could be used with certain topical agents that are usually applied in their crystalline state. With freezing, the crystals will fracture causing the agent to be more accessible for reactivity on rewarming. In the case of benzoyl peroxide, the crystals would crack and this would allow more surface area and more benzoyl peroxide to react with other topical agents like tertiary amines.

Various lasers have different wavelengths and penetration capabilities. In the example of acne, the three main targets of lasers are the bacteria (Propionibacterium acnes), the sebaceous glands, and the acne biofilm. Indeed, the various layers would have significance in altering the acne biofilm at different depths within the hair unit. Inasmuch as P. acnes can live in aerobic and anaerobic conditions, different lasers with different penetration capabilities may be advantageous in different people with these topical agents. By using topical agents (especially those which form benzoyl peroxide radicals) in association with lasers would seemingly offer a greater effect on the acne biofilm, and lead to greater and more lasting improvement in patients' acne status.

An example of a condition one may opt to apply prior to (or during, or after) laser (and other forms of light therapy such as light emitting diodes) treatment is for hyperhidrosis of the palms and/or axillae. In this case, one would put aluminum chloride hexahydrate (or a similar compound) prior to laser therapy so that it is absorbed into the skin. This topical causes constriction of the isthmus that allows sweat to surface from the eccrine gland. Laser fibrosis (or scarring) can further narrow this passageway of sweat, alleviating the clinical condition. One might additionally apply a polytrap polymer (like Acrysorb, a acrylate polymer with microparticles that absorb excess oils) to the skin prior and/or with and/or after treatment with the laser.

Another example of a product that would be helpful to apply prior to (during and/or after) laser (and other forms of light therapy such as light emitting diodes) therapy would be products containing salicylic acid, glycerol, ethylene glycol, polyethylene, and sucrose. Similar to methods used to preserve sperm by cryogenics, these substances increase the tolerance of cells to dehydration, maintaining tissue viability. Thus, these agents would lessen the collateral heating and damage from laser therapy. This topical preparation could be used with other topical agents, such as numbing agents or topical prescription medicines.

By using lasers with benzoyl peroxide as well as other topical medications, one expands not only the use of the topical agents, but also of laser (and other forms of light therapy such as light emitting diodes) usage. Moreover, some diseases, which presently require oral therapies, may be arrested with topical therapies and lasers. This therapy may also significantly decrease the number of laser (and other forms of light therapy such as light emitting diodes) treatments and recurrence of acne and other dermatologic disease states. An example would be with tinea capitis. This condition presently requires oral treatment because the fungus lives in the hair unit under the skin. With certain lasers, we can activate the topical agent (for example, benzoyl peroxide with a tertiary amine) and also temporarily exfoliate the hair. Without dead hair to live off of, the fungus leaves the hair unit, and the epidermal tissues can then grow a new hair without fungus in the environment. In a similar manner, one could treat onychomycosis without oral therapy.

Another ramification in using lasers (and other forms of light therapy such as light emitting diodes) with topical therapies is that one might be able to target specific skin organs not normally controlled perfectly by either modality by itself. Returning to acne, one might use a laser with with specific topical medications (such as tazoratene and/or benzoyl peroxide/tertiary amine) to suppress the sebaceous gland. Certain lasers have been shown to shrink sebaceous glands temporarily; however, these topical agents used in conjunction with lasers, may lead to a prolonged effect on the sebaceous gland and the acne biofilm.

Additionally, as mentioned above, lasers (and other forms of light therapy such as light emitting diodes) may prove a helpful adjunct to assist (or augment, or activate) topical chemotherapeutic agents against skin proliferation and skin tumors (and systemic tumors). In the case of benzoyl peroxide and tertiary amines, imagine these agents when exposed to laser rays emit benzoyl peroxide radicals that toxic to cancer cells.

Besides benzoyl peroxide, there are numerous other topical agents that could be improved and/or activated by means of laser (and other forms of light therapy such as light emitting diodes) accentuation. Different chemicals may be activated by different wavelengths. Also the fluence, power, type of laser, and focal points might have to be adjusted with the various chemicals one is activating. Thus, the therapeutic activity of some topical agents such as hydroquinone and azelic acid for hyperpigmentation would be improved merely by heating the skin with lasers. Also on point, many of the topical preparations are not totally in solution when applied to the skin. This list of products, which may improve therapeutics and/or solubility by applying laser waves to them on the skin surface, include (but not limited to) pramoxine hydrochloride, metronidazole, triple antibiotic ointment, urea, and sulfacetamide.

Additionally one may apply a topical agent such as nitrates (such as nitroglycerin), capsaicin or minoxidil prior to laser (and other forms of light therapy such as light emitting diodes) treatment for vascular diseases. These products (or others) may make vessels enlarge (or be more sensitive to laser treatment and the like) and improve treatment of vascular lesions (or reduce the number of treatments needed) with lasers. I note that minoxidil is a tertiary amine and may also be benefited by the co-administration with benzoyl peroxide.

Lasers (and other forms of light therapy such as light emitting diodes) may also prove beneficial for certain parasitic diseases such as scabies, head lice, and larvae migrans. They may prove beneficial by themselves (for example for head lice), or may prove helpful in augmenting the abilities of antiparasitic agents (to which there is growing resistance), or may activate proteases (to damage the proteinaceous lice eggs) or chitinases (to damage the parasites' exoskeleton).

Lasers (and other forms of light therapy such as light emitting diodes) may also prove helpful in enhancing the penetration of topical immunomodulators deeper into epidermal and dermal tissues (immunomodulators have been discussed in one of our previous patents).

This concept is to be expanded for the use of all types of lasers (and other forms of light therapy such as light emitting diodes) for all other body organs for all other uses in humans and animals. For example, one may use a laser with benzoyl peroxide and a tertiary amine applied to (or injected into) a tumor in the abdomen.

Presently, lasers are not used with any topical agents save for anesthetic agents to reduce the pain for the patient. There has been some concern that one should not apply anything on the surface that might lead to light wave reflection. This was of significance when protective eyewear was not standard procedure, but using topical agents with lasers (and other light sources), may have therapeutic advantages. Moreover, not only may lasers be used in consort with topical medications, they may be used to excite or activate certain topical agents for improve therapeutic function and advantage. In such case, the laser (or light source) would have additive, or possibly synergistic biological effects when used in concert with topical agents.

Inasmuch as some lasers hit water, one could make the benzoyl peroxide preparations and tertiary amines and transitional metals either very low or high in water content. Decreasing the water content also increases the potency of benzoyl peroxide.

Light therapy may have an additional benefit when used with some topical agents. For example, black light may assist in the treatment with some antibiotics, like tetracycline, in making sure that good coverage of the involved areas was achieved (as tetracycline fluoresces). Of note, tetracycline has a tertiary amine. In such a scenario, benzoyl peroxide, and a trace metal might be added to the skin, causing color changes that should coincide with therapeutic treatment. This would make ensure that coverage of involved areas was achieved, but that the chemical reaction needed for optimum results, is taking place.

The treatment may be used in compliance with this patent in which the medication is in the form of a dressing or foam, which is then aided or activated by the laser of light treatment.

Conditions by which this patent may be utilized include:

Disorders of collagen, elastin, and ground substance (such as stretch marks)
Diseases of the subcutaneous tissue
Neurofibromas
Blistering diseases (such as epidermolysis bullosa, pemphigoid)
Urticaria Pigmentosa

Diseases of cornification (such as ichthyosis)
Disturbances of melanin pigmentation (such as lentigines, melasma, acanthosis nigricans, vitiligo)
Acne and acneiform dermatoses
Diseases of the apocrine and eccrine sweat glands
Hair disorders
Disorders of the nails
Cutaneous mucinoses and amyloidosis (such as myxoid cysts)
Xantholasma and other skin storage diseases
Sarcoid
Benign and Malignant Tumors of the skin
Linear epidermal nevus
Tumors of epidermal appendages
T cell lymphomas
Urticaria (for example, diphenhydramine HCl is a tertiary amine, and may be more valuable once further deployed by the addition of benzoyl peroxide and light)
Eczema
Pruritus
Diseases of the oral cavity
Bacterial, fungal, and viral infections and for sterilization
Use with cytotoxic agents
Chemical peels
Improve the permeability of the skin
Diminish skin aging and its effects on the skin
Granulomatous skin diseases
Papulosquamous eruptions and exfoliative dermatitis
Warts, molluscum and other skin growths
Diseases os the mononuclear phagocytic system, the so-called reticuloendothelial system (such as leishmaniasis)
Connective Tissue Diseases

Within the context of this invention is the use of the light emitting diode (LED) with topical agents for dermatologic disease states. LED emits incoherent monochromatic light which has shown promise in wound healing and pain, but we believe that it could also be applicable to use in consort with benzoyl peroxide and other topical agents in the treatment of numerous dermatologic states. A few additional comments regarding this form of energy in the context of our work follow. These LEDs of course emit energy. LEDs have been helpful in wound care without any specific reason found medically. However, it may allow the skin to have a more even pattern of electrical waves, thereby correcting what I call skin electrical shorts caused by disease or insult. The neurological system is connected with the immunological system of our bodies, and thus LED would affect our immune response as well. Thus, LED provides an electroluminescence to the skin. Using plant analogy, LED (or their general wavelength of light) is needed for many forms of plant growth. LED has been shown to upregulate certain tissue regenerating genes as well as increase fibroblast proliferation. The possible synergy of LED with benzoyl peroxide can be inferred by both increasing polymerization. Both also have potential of producing cytotoxicity and oxidative stress. I should again note that this form of energy (similar to lasers) has only been used in photodynamic therapy which is where one uses visudine (a benzoporphyrin), 5-aminolevulinic acid, or similar compound. Photodynamic therapy involves the selective retention of a photosensitizer that upon activation with light mediates tumor cell destruction via the production of singlet oxygen.

Benzoyl peroxide has been used for acne since the 1960s in various over-the-counter and prescription creams, lotions, and washes. The December issue of LANCET (2004;364:2188-95) has spurned more interest in benzoyl peroxide, as it was shown to be the most cost-effective acne regime. Indeed, it is the only active ingredient in ProActiv Solution's 3-step therapeutic process. One could call our over-the-counter product a new and improved, second-generation proactive solution infused with the advantage of applied science with clinical benefit.

Our concept is to make a more effective benzoyl peroxide. This is accomplished by accelerating the conversion of benzoyl peroxide into its more active state, which is called a benzoyl peroxide radical. This chemical transformation can be accomplished by means of a stimulant, an accelerator, and modification of the vehicle.

The stimulant is a tertiary amine. Chemically, a tertiary amine is a substance that has a type of nitrogen molecule somewhere in its structure. This readily converts benzoyl peroxide into a benzoyl peroxide radical. Although this concept is poorly appreciated in medicine (indeed, we have the only medical publications on this reaction in dermatology), it is well appreciated in other scientific fields. For example, benzoyl peroxide radicals are used to initiate a process called polymerization that is used for bone cements and for mending cracks on the wings of airplanes. The beauty of this reaction of forming benzoyl peroxide radicals is its magnitude, quickness, and therapeutic potential.

Several tertiary amines are available for topical use. Examples include prescription antibiotics (erythromycin and clindamycin), as well as over-the-counter antifungal agents, (terbinafine and butenafine).

Of note, there are social, epidemiologic, and medical concerns over the development of antibiotic-resistant organisms as well as an increased risk of breast cancer due to prolonged use of oral and topical antibiotics. Our treatment using a topical antifungal agent with benzoyl peroxide eliminates these concerns.

Accelerators for this reaction between benzoyl peroxide and a tertiary amine are trace metals, such as zinc. These agents reduce the energy level needed for the production of benzoyl peroxide radicals.

There are several methods by which one can alter the environment to maximize the power of benzoyl peroxide. One method is to warm the skin prior to (during and/or after) its application. This could be done with light, warm soaks, or lasers. Additionally, benzoyl peroxide is also more active when the base (i.e. vehicle, other constituents in the cream or gel) in which one places the active ingredients of the reaction has a low water content. For example, water-less sprays can be cosmetically elegant and fulfill this need. One can also add PEG to the vehicle to increase the activity of benzoyl peroxide. The idea includes that we can affect the solubility and reactivity and efficacy of benzoyl peroxide by altering the base in terms of solvent polarity, water content, and amount of PEG400 and other PEG units.

Thus, to maximize benzoyl peroxide radical formation and clinical results, one of our preferred present methods of treating acne is:
1. Soaking the face in warm water. (Heat will aid in the chemical reaction involving benzoyl peroxide).
2. Applying the tertiary amine (which might be erythromycin, clindamycin, terbinafine, or butenafine) in the form of a spray or solution.
3. Applying zinc in a spray or solution on top of the tertiary amine.
4. Applying the benzoyl peroxide in a gel (or cream) form in an alcohol base on top of the two sprays directly onto the skin surface. (Many benzoyl peroxide radicals will form instantaneously when the products are combined. Benzoyl peroxide is in a crystalline state so there will continue to be some radical formation, but to a lesser degree after the initial mixing.)

We are finding this combination helpful in treating fungal infections (jock itch, athlete's foot, nail fungus), wound care, soaps, and cleansers. For example, our studies with University Hospital revealed that using this combination lead to additive activities against the vast majority of yeasts tested and expanded bacterial coverage. Indeed, this combination treatment should improve the penetration abilities into nails (onychomycosis) and hair. Thus, one would find soap with our combination better at removing bacteria, virus and fungal elements than present available cleansing agents. This patent has numerous applications for both human and animal needs.

We have developed the concept of using benzoyl peroxide with a stimulant such as tertiary amines with an activator (such as zinc) to lower the energy level for the reaction to take place. We have also previous discussed the use of having a vehicle low in water to make the benzoyl peroxide more potent.

In this we are including more possible uses of this concept. The other uses would be treating the various body areas: vagina, bladder, urethra, rectum and anal region, mouth, ears, gastrointestinal, skin, joints, throat, any wounds (including internal and those related to trauma), stomal care, pre and post (and possibly during) surgical care, throat, lungs, and nasal areas. This embodiment also has applicability in animals.

The uses would be to protect against, treat for, or attempt to reduce the incidence of infection including bacteria, fungal and viral origin. It could be used as a flush, gel, cream, solution, or any other method of putting these agents into the regions needed. It could be used separately or with other topical or oral agents. It might be used in consort with lasers or some other energy source such as heat, light, etc. It could be used to eliminate certain pathogens as a sterilizer pre-surgery on the skin. I might be used to joint capsules or flushing the joint during surgery.

Examples of using this combination would be in a vaginal suppository for non-specific vaginitis, in a rectal insertion for diverticulosis, and a nasal spray for carriers of methicillin-resistant Staph. aureus.

Benzoyl peroxide (with or without activators and accelerators) may be useful in consort with all forms of light therapy, including lasers, light emitting diodes, blue light, and all other forms of phototherapy. Moreover, it can be used in all forms of products and technologies in the treatment of soft tissues via all energy-based modalities including cryogenic energy, hydraulic energy, laser energy, magnetic energy, mechanical energy, microwave energy, radiation energy, radiofrequency energy, thermal energy, vibrational medicine, and ultrasonic energy.

Expanding an item in a previous patent, we would be utilizing the benzoyl peroxide/tertiary amine idea in the use of make-up, shampoos, sun screens, acne washes and all categories of cosmetic items and soaps and cleansers. For example, one might use a teriary amine in the foundation and benzoyl peroxide in the wash, toner or rinse. Such products could be OTC or prescription.

One might want to apply the benzoyl peroxide first to the skin, followed by the application of the tertiary amine (such as erythromycin) in a buff-puff, pad, or loofah and then scrub the two together on the skin surface. This could also be done in the reverse fashion. This could have applicability for acne, acne washes, and other skin conditions. In short, there are innumerable ways in which one can put these two products together on the skin, either for acne, cleansing, protection or treatment for MRSA, or the like.

The benzoyl peroxide/tertiary amine concept could also be used in a fashion more conducive in removing plugged pores. We have shown in our study that this combination is more unplugging than benzoyl peroxide by itself (Proactiv Solution) and in the prescription products (such as Benzaclin). To improve this, we would be using them in tape strips, something similar to Biore strips, except these would be medicated. For example, the benzoyl peroxide would be applied to the skin, followed by a tertiary amine (such as erythromycin) incorporated into a tape. The combination would then come together on the skin surface. One might want to apply heat or light (to increase the reaction). Then when one strips off the tape, many of the plugs would be loosened in the process. Light or heat may help the reaction to take place. This embodiment of the invention and the others described are also applicable to use in animal care.

Using medicated products to assist removal of blackheads. Similar to Biore and to our product discussed above using benzoyl peroxide application to the skin followed by a tape with a tertiary amine in it to affected areas, one could use this concept with other medicated, prescription agents either applied before the tape application, or have the medication incorporated into the tape.

Other devices would use magnets to gently asist express of the plug pores. A suction device would also assist in their removal. A medical device to assist in this unplugging action is part of this idea.

In terms of a condition called “Pruritic Scalp”, patients experience itchy scalp with (or without) clinical signs of dandruff, seborrheic dermatitis, head lice, or psoriasis. In most patients, the scalp just itches for no obvious reason on clinical examination. It may well be a condition involving the skin nerves in the area.

The present invention uses a topical agent to treat this condition which includes several antipruritic agents combined together. Thus, for example, one would include lidocaine, camphor, phenol, antihistamine and a steroid into the container. A vitamin D compound (such as cod liver oil) may also be useful.

The products can be used in various combinations and in several ways. For example, one or more of the anti-itch products may be in the shampoo, while another in the conditioner, rinse, cream (like Brylcream), in a spray (that one might rub into the scalp), a foam, or in any formulation (of any kind) in which a substances reaches the scalp skin. The products can be in OTC or prescription formulations.

Also, one may want to eradicate possible infective agents as a cause of the itch. One can do this by using benzoyl peroxide and a tertiary amine as a double application with shampooing. There are various ways in which this could be applicable, for example, one could use the tertiary amine in the shampoo and then use the benzoyl peroxide as a secondary wash to apply on top of the other shampoo. This then would be an extension of our previous work with benzoyl peroxide and tertiary amines.

The addition of light may prove helpful. Thus, we include any medical device which allows light to reach scalp skin, or allows the hair to circulate so the light source can get to the various portions of the scalp skin.

Dihydroxyacetone (DHA) is a triose carbohydrate that is used in the cosmetics industry as a tanning substance and also in fungicides. To improve the tanning process with DHA, one could use a formulation with amino acids (and/or proteins) in an form of topical delivery system prior to (during, or after) the DHA application. This would allow the skin to contain more amino acid for binding to the DHA.

Additionally, one could put all of the various formulations discussed above (the DHA, or the amino acid preparation, the protein preparation) in a polymer base. this allows deposition of more product within the outer skin layers and loads it there. For example, one could make the amino acid preparation in a polymer base which dries on the skin surface.

One might use a topical preparation after the application of the DHA. such a product might contain a moisturizer, and/or a sunscreen, and/or more DHA (to constantly even out the tone to those areas which are lighter), and/or amino acids to help DHA binding. Indeed, one might use several products after the initial DHA application: for example, one with high DHA to areas that need more pigment, and one with minimal to no DHA (or even a keratolytic agent to remove some of the over-pigmented skin) to areas that are darker than the surrounding skin.

In terms of sunless tanning, one could use the benzoyl peroxide and tertiary amine as an application of exfoliative purposes and for cleaning the skin of toxins and infectious agents prior to the application of the substances (or sprays) with dihydroxyacetone. For example, one might use the benzoyl peroxide in some emulsion form, followed by a tertiary amine (like erythromycin), in the form of a spong or loofah scrub. As suggested by sunless tanning, one should exfoliate prior to sunless tanning, and we would be providing the best method for exfoliating and cleansing the skin of possible pathogens and other agents (fungus) which may pose a problem for obtaining maximum results with sunless tanning. Such a method could still be used in consort with other exfoliators, if desired. This treatment with benzoyl peroxide and tertiary amines would also assist against tinea versicolor, which often makes for a mottled skin appearance.

One could also use one of various keratolytic agents (many of which can be OTC or Rx) as the exfoliator prior to DHA application. These products could contain DHA and/or amino acids or one could have a step process to maximize one's results with DHA. This would be similar then to Proactiv Solution, in which one applies three products, one after another. These DHA and amino acid products could also be used in consort with any of the anti-aging, anti-wrinkling products from retinoids to hyaluronic acid. They could also be used with skin fillers.

The addition of heat and/or light may help with the reaction of DHA attachment to the proteins in the skin.

These ideas could also apply to coloring of the hair and/or nails.

Benzoyl peroxide and a tertiary amine can also be used in combination with a keratolytic agent to assist in the removal of seborrheic keratoses and other skin abnormalities such as hyperkeratotic states (such as warts, psoriasis callouses, skin tumors, corns, molluscum, acne, tumors, lichen simplex chronicus and the like. the product would assist in the exfoliation of these skin abnormalities. Such a product may also assist in the treatment of eschars, scabs, wound healing, wound care, pre-skin cancers, skin cancers, xerosis, skin cracks and fissures, dermatitis, eczema, and benign keratoses. The tertiary amine/benzoyl peroxide would reduce the number of possible pathogens as well as assist in the debriding. Any suitable ketatolytic agent can be used, such as alpha-hydroxy acids, propylene glycol, retinoids (including retinoic acid, adapalene, tazarotene, and the like), sodium lauryl sulfate, proteinases, salicylic acid, vitamin D analogs (such as Dovonex), ichthyol, coal tar, and any other product that is keratolytic or proteolytic.

In some embodiments of the invention, the described treatment method includes the use of a peroxide, such as benzoyl peroxide, with any type of tertiary amine, and in some embodiments it includes all tertiary amines except erythromycin and clindamycin.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. A method of treating a skin condition comprising first applying to the skin a combination of a peroxide and a tertiary amine and then applying a light energy to the skin.
